(19) United States
(12) Patent Application Publication
(10) Pub. No.: US 2024/0418735 A1
(43) Pub. Date: Dec. 19, 2024

(54) Methods and Kits for Determining Insulin Resistance Treatment

(71) Applicant: Scimar Ltd., Dauphin (CA)

(72) Inventor: Wayne W. Lautt, Gibbons (CA)

(73) Assignee: Scimar Ltd., Dauphin (CA)

(21) Appl. No.: 18/706,051

(22) PCT Filed: Nov. 4, 2022
(86) PCT No.: PCT/CA2022/051636

§ 371 Date: Apr. 30, 2024

Related U.S. Application Data
(60) Provisional application No. 63/275,786, filed on Nov. 4, 2021.

(51) Int. Cl. G01N 33/74; A61K 31/198; A61K 31/27

(52) U.S. Cl. CPC G01N 33/74; A61K 31/198; A61K 31/27; G01N 2333/62; G01N 2800/042; G01N 2800/52

(57) ABSTRACT

A method for determining whether an individual with prediabetes, AMIS, type 2 diabetes, gestational diabetes, insulin resistance, or impaired glucose tolerance will respond to a bethanechol and N-acetylcysteine treatment is taught. Blood samples are taken on a placebo day and on a test day. If the administration of bethanechol and N-acetylcysteine resulted in a reduced postprandial increase in glucose and insulin, this indicates that the drug could be beneficially used.

### CROSS REFERENCE TO RELATED APPLICATIONS

The present application claims priority to U.S. provisional application No. 63/275,786, filed Nov. 4, 2021.

### FIELD OF INVENTION

The present invention relates to a method, use, and kit for determining an individual's responsiveness to treatment of insulin resistance, and in particular, to determining an individual's responsiveness to treatment for Absence of Meal-Induced Insulin Sensitization (AMIS)

### BACKGROUND

AMIS (Absence of Meal-Induced Insulin Sensitization) is the term used for a metabolic dysfunction of the uptake, storage, and management of the nutrient energy from meals. AMIS represents the missing link in the development of prediabetes, obesity, and type 2 diabetes. The science of AMIS, its diagnosis, prevention, and therapy has advanced since our first report (Lautt 1996) of the previously unsuspected hormone, HISS (Hepatic Insulin Sensitizing Substance), also known as hepatalin.

MIS (Meal-induced Insulin Sensitization) is the healthy response to a meal whereby insulin stimulates secretion of the hormone, hepatalin, previously referred to as HISS, from the liver. Hapatalin acts on muscle, heart, and kidneys, but not liver, guts, or fat. Up to 80% of the uptake of nutrient energy in the form of glucose is taken up and stored as glycogen in these organs. In a healthy metabolic state, insulin (33%) and hepatalin (66%) allows proper nutrient partitioning. Nutrient partioning to fat (proporation of glucose uptake in liver and fat and nutrient partioning to muscle (proporation of glucose uptake in muscle, heart, and kidney) is carried out.

Impaired signals result in reduced hepatalin production leading to postprandial hyperglycemia with compensatory elevation of insulin secretion and a shift in partitioning of nutrient energy away from the muscle and towards lipids. If hepatalin secretion is impaired, insulin loses about ⅔ of its impact on glucose uptake (Patarrao et al, 2008). Postprandial hyperglycemia is increased and prolonged; insulin secretion more than doubles and insulin shifts the majority of nutrient energy partitioning to the liver and fat. When glycogen stores in the liver become saturated, the excess nutrient energy is stored as triglycerides, which results in hyperlipidemia. Free radical stress in increased and the vasodilator action of hepatalin is absent.

AMIS syndrome begins as postprandial elevations in glucose, insulin, fat and free radical stress. A predictable chronology of dysfunctions results from prolonged AMIS. In AMIS, insulin to hepatalin ratios become closer to 90% insulin and 10% hepatalin.

AMIS syndrome: If AMIS becomes chronic, the shift in nutrient partitioning results in a predictable progression of pathologies known to be associated with obesity and diabetes, including elevated blood levels of triglyceride, cholesterol, insulin, glucose, free radicals, and increased blood pressure and vascular dysfunctions in heart, kidneys, eyes, and feet (hepatalin is a powerful dilator of blood vessels). The pathologies are components of the AMIS syndrome.

Regulation of hepatalin secretion: hepatalin secretion from the liver is absent in the fasting state when storage of nutrient energy is completed, and the energy stores are being slowly used up until the next meal. The presence of a mixed meal in the stomach activates two feeding signals that are sent to the liver. One signal is mediated by activation of hepatic parasympathetic nerves. The sensory signal from the stomach is sent to the brain and is relayed to the liver via the hepatic branch of the vagus nerve. The nerve secretes the neurotransmitter, acetylcholine, which acts on muscarinic receptors in liver cells. The second signal is a chemical signal resulting in nearly doubling of hepatic glutathione (GSH) levels. GSH is essential for the production of hepatalin. The signals are permissive and synergistic in that both signals are required but they act only to mimic the mechanisms that allow insulin to stimulate secretion of hepatalin from the liver. The 2 signals can be disrupted by oxidative stress, diets high in sugar, obesity, many drugs including alcohol, emotional stress, and normal aging. Neither signal is functional in the diet-induced model of prediabetes nor gestational diabetes.

AMIS therapy: The two signals can be mimicked by a combination of two drugs. Preclinical pharmacology was extensively studied as pure basic research using a range of cholinergic drugs and GSH enhancers. Two repurposed drugs with known safety records were found to be superior. Bethanechol activates muscarinic receptors, mimicking the nerve signal. N-Acetyl Cysteine is rapidly incorporated into the GSH molecule, resulting in GSH levels increasing to healthy postprandial levels. The presence of both permissive signals renews the ability of insulin to stimulate hepatalin secretion and restores the healthy balance of nutrient energy partitioning. However, the administration of bethanechol in combination with N-acetylcysteine is not effective in providing relief of these complications or in improving the hepatalin response in all patients. Thus, there is a need for a way to diagnose whether the bethanechol and N-acetylcysteine treatment would be effective in improving the hepatalin response in a given patient.

### SUMMARY OF THE INVENTION

In a first aspect, the present invention provides a method for diagnosing whether an individual with prediabetes, AMIS, type 2 diabetes, gestational diabetes, insulin resistance, or impaired glucose tolerance will respond to a bethanechol and N-acetylcysteine treatment, the method comprising:
on a test day:
   taking a first blood sample on the test day from the individual; administering bethanechol and N-acetylcysteine to the individual;
   administering a meal to the individual;
   waiting a period of time; and
   taking a second blood sample on the test day from the individual;
on a placebo day:
   taking a first blood sample on the placebo day from the individual;
   administering a placebo to the individual;
   administering the meal to the individual;
   waiting the period of time; and
   taking a second blood sample on the placebo day from the individual;
      analyzing each of the blood samples for one or more of a glucose level and an insulin level; quantifying a test increase in glucose and/or insulin between the first blood sample on the test day and the second blood sample on the test day; and
      quantifying a placebo increase in glucose and/or insulin between the first blood sample on the placebo day and the second blood sample on the placebo day;
      wherein a reduced increase in glucose and/or insulin on the test day as compared to the placebo day indicates that the individual will respond to the bethanechol and N-acetylcysteine treatment.

In a another aspect, the present invention provides a diagnostic kit for use with blood sample drawing equipment and blood analysis equipment for diagnosing whether an individual with prediabetes, AMIS, type II diabetes, insulin resistance, or impaired glucose tolerance will respond to a bethanechol and N-acetylcysteine treatment according to the method described above, the kit comprising: a first composition comprising bethanechol and N-acetylcysteine; a second composition comprising an inactive substance; a first meal; and a second meal, the second meal being identical to the first meal.

### BRIEF DESCRIPTION OF THE FIGURES

FIG. 1 is a graph showing BENAC (bethanechol and n-acetyl cysteine) synergistic treatment of AMIS.

FIG. 2 is a diagram showing the hepatalin synthesis hypothesis.

FIG. 3 is a flowchart illustrating a method for diagnosing whether an individual will respond to a bethanechol and N-acetylcysteine treatment according to an embodiment of the present invention.

### DETAILED DESCRIPTION

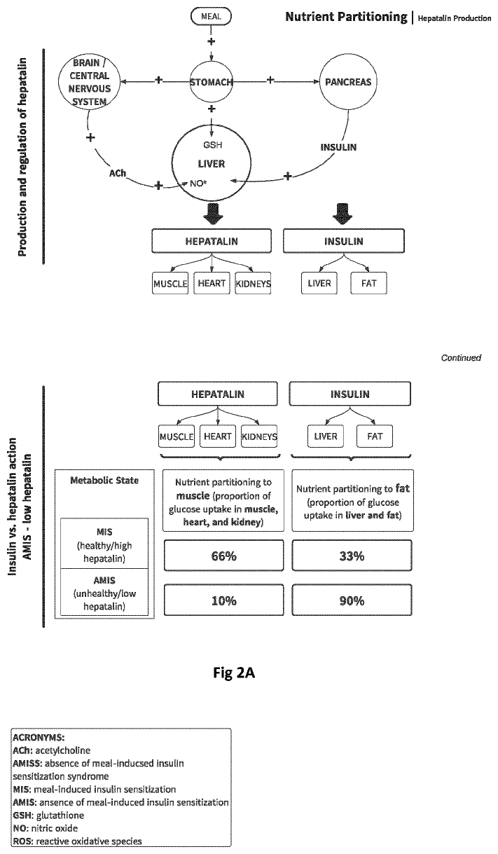

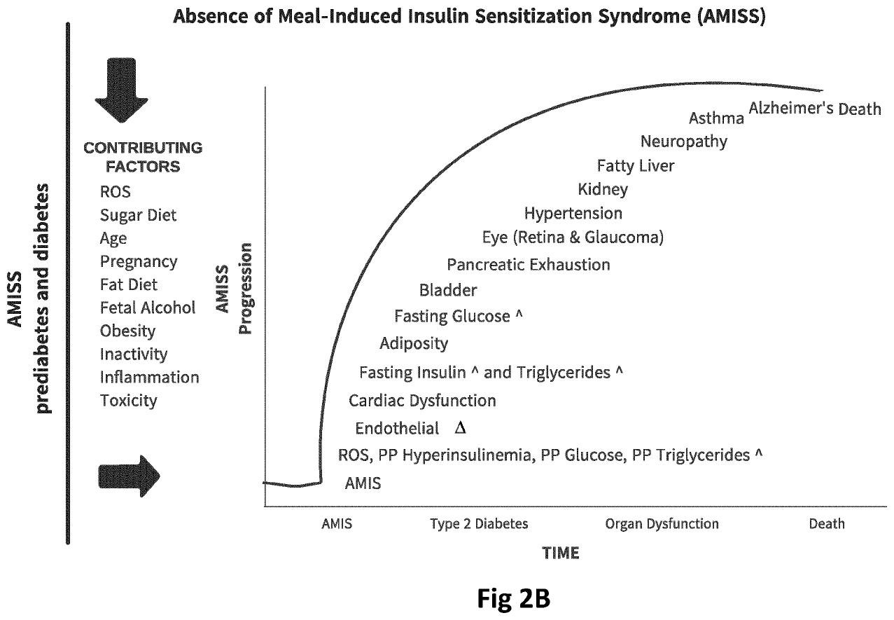

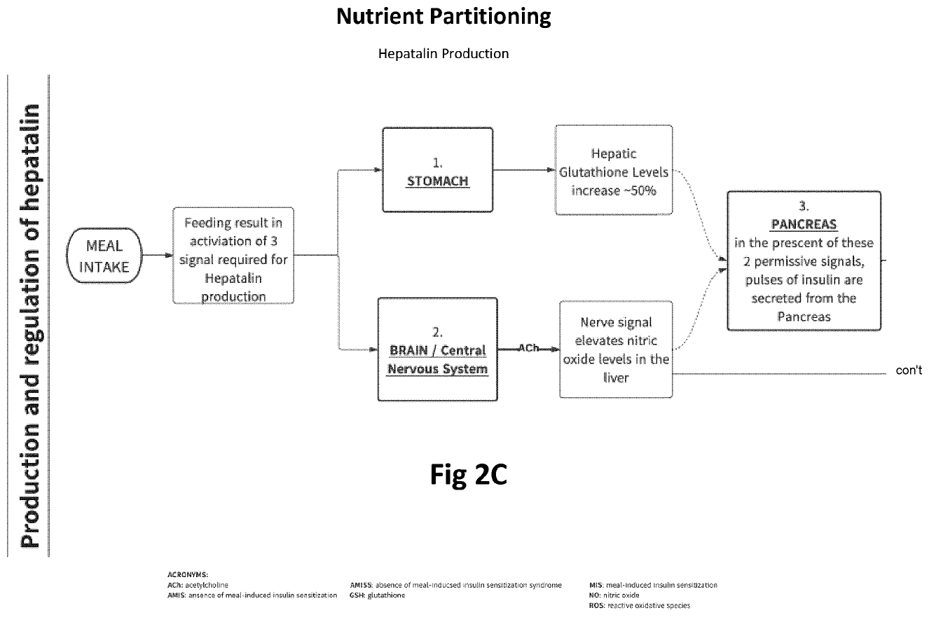

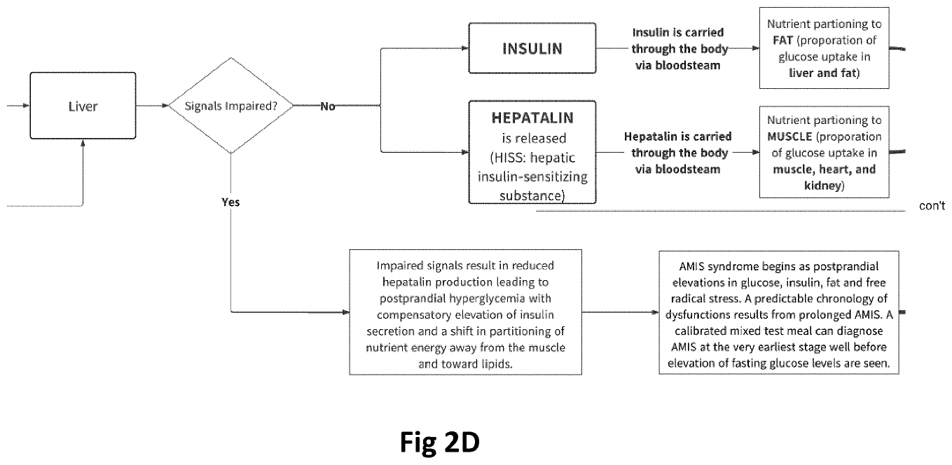

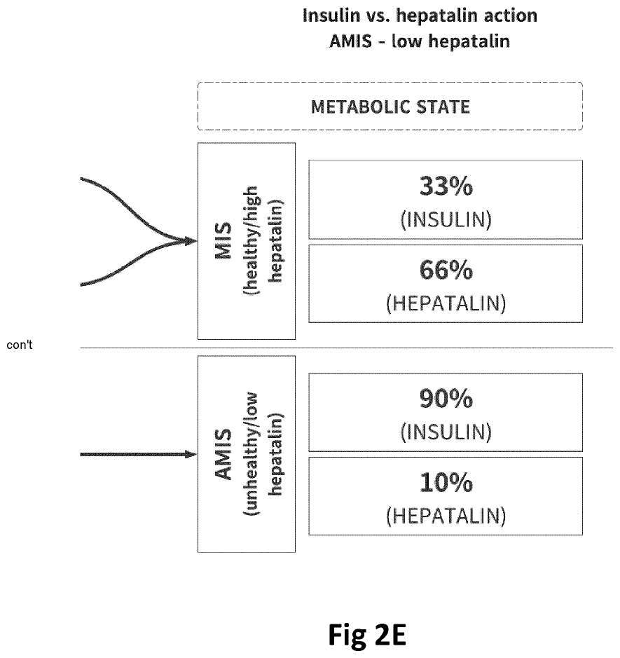

While the present invention is not limited to a particular model or mechanism of action, it appears the response to feeding results in the release of acetylcholine which activates muscarinic receptors in the liver. This activation leads to increased production of nitric oxide which stimulates guanyl cyclase activity, resulting in increased levels of cyclic guanosine monophosphate which acts in stimulating the release of hepatalin from the liver. Meal-induced insulin secretion (MIS) results from insulin-induced secretion of hepatalin which can only occur if the liver receives two permissive feeding signals. Absence of either signal blocks the ability of insulin to trigger hepatalin secretion. The combination of bethanechol and N-acetylcystelne (BENAC) mimics the 2 signals. As shown in FIG. 2, rats treated with bethanechol and N-acetylcystelne showed the greatest potentiation in RIST (Rapid Insulin Sensitivity Test) as compared to the control rats. The combination therapy was more effective than either bethanechol or N-acetylcystelne alone for restoring insulin sensitivity in sucrose fed rats.

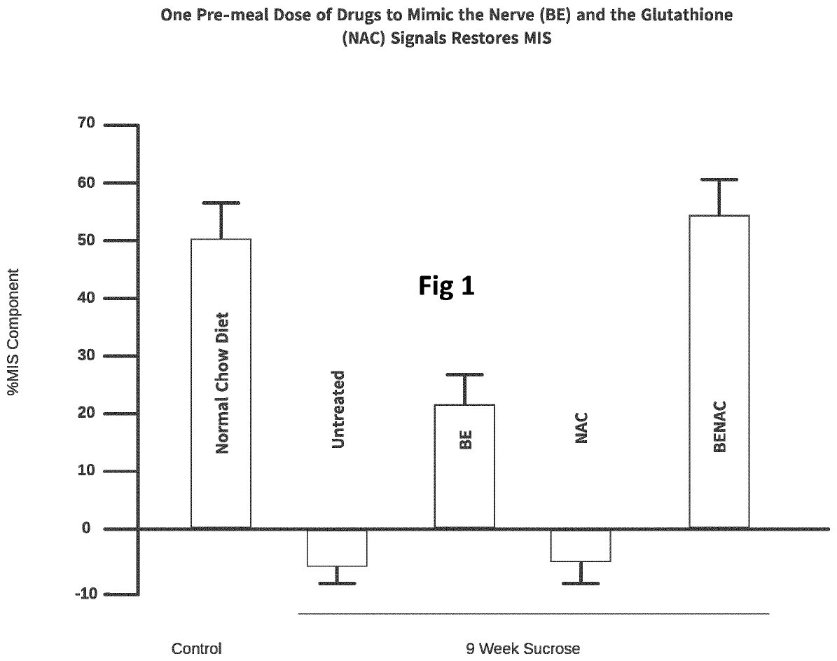

FIG. 1 is a chart showing BENAC synergistic treatment of AMIS. The % MIS (hepatalin/insulin action×100%) calculated from the RIST index in 24 hour fasted anaesthetized rats determined before and 90 minutes after intragastric injection of a mixed liquid meal in a normal control group and in four groups that had been treated with nine weeks of sucrose supplementation. The sucrose diet completely blocked MIS. Bethanechol (BE) produced a modest restoration of MIS in the diabetic model. N-acetyl cysteine (NAC) elevated GSH levels to that seen after feeding but had no effect on the RIST index. The combination of the two feeding signals (BENAC) resulted in a full restoration of MIS. Increasing the doses of either NAC or BE restored MIS to levels seen after feeding but not above. This model demonstrates that in sucrose-induced AMIS both the nerve and GSH signals were absent but were able to be restored by mimicking these signals with pharmaceuticals. The synergistic permissive nature of the feeding signals is demonstrated.

However, it has been further discovered that the bethanechol and N-acetylcysteine treatment may not be effective in improving the hepatalin response in all patients depending on the cause of their insulin resistance and/or a related disorder. For example, it has been found that the bethanechol and N-acetylcysteine treatment was not able to help restore hepatalin secretion in rats with Absence of Meal-Induced Insulin Sensitization (AMIS) when the insulin resistance was a result of blood loss, chronic inflammation, and/or stress. Other causes that block HISS secretion in an individual may also not be amenable to the bethanechol and N-acetylcysteine treatment.

FIG. 2 is a diagram showing the hepatalin synthesis hypothesis. The AMIS syndrome is the progressive, predictable, chronological accumulation of signs and symptoms of homeostatic disturbances caused by absence of hepatalin action after each meal. Absence of hepatalin action in the fasted state is appropriate, but if it is not rapidly reversed after a meal, leads to postprandial increases in glucose, insulin, lipids, and reactive oxygen species (ROS). AMISS progresses to prediabetes, obesity and type 2 diabetes, and associated dysfunctions in major organ systems. By the time fasting glucose is elevated the AMISS is well underway. The specific order of appearance of dysfunctions is an estimate that requires verification. A variety of contributing factors lead to suppression of hepatalin secretion. Treatment options vary with the duration and extent of AMISS. ROS=reactive oxidative species, PP=postprandial. (Figure modified from Lautt et al., 2010, Can. J. Physiol. Pharmacol. 88: 313-323).

Hepatalin production is regulated by the synergistic permissive action of feeding signals from the stomach activating hepatic cholinergic muscarinic stimulation of nitric oxide synthase and generation of cGMP. Normally, feeding results in the activation of three signals required for hepatalin production: glutathione, nitric oxide, and a pulse of insulin. Hepatic glutathione levels increase by about 50%. Nerve signals elevate nitric oxide levels in the liver. These signal the pancreas to release pulses of insulin. In the presence of both synergistic, permissive feeding signals, pulses of insulin stimulate secretion of pulses of hepatalin. The degree of production of hepatalin determines the ratio of partitioning of nutrient energy storage as glycogen or lipids.

The present invention relates to a method, use, and kit for determining an individual's responsiveness to treatment of insulin resistance, and in particular, to determining an individual's responsiveness to treatment which modulates hepatic sympathetic and parasympathetic action, and in particular to diagnose abnormal postprandial processing of nutrients and to determine responsiveness to one premeal dose of diagnostic which then becomes identified as a suitable therapeutic, i.e a theranostic.

The theranostic concept is based on clinical diagnostic tools validating the diagnostic to be used as a therapeutic. A calibrated mixed test meal can diagnose AMIS at the very earliest stage well before elevation of fasting glucose levels are seen.

Diagnostic: Both the nerve and the GSH signal are absent in diet-induced diabetic rats. Rats on a sucrose supplemented diet had complete absence of hepatalin action. AMIS had been developed by week 2 and the condition was maintained for an additional 7 weeks. One dose of the combination of bethanechol and n-acetyl cysteine (BENAC) given before a test meal fully restored the ability of insulin to stimulate hepatalin secretion. However, given separately they were ineffective thus demonstrating a negative synergistic regulatory mechanism (Lautt et al., 2011). The same results were obtained with a diet-induced gestational diabetic rat model (PhD thesis N. Lovat). A diagnostic has thus been demonstrated showing that AMIS was detectable using a test meal and that the mechanism of the dysfunction was with the two feeding signals and could be acutely treated by mimicking the feeding signals with BENAC.

Therapeutic: A clinical trial of 12 weeks duration was a double-blind placebo-controlled testing of the effect of the drugs administered orally 1 hour prior to each meal. The subjects (n=34 treatment group, 36 placebo group) were non-insulin dependent type 2 diabetics diagnosed at least 3 months prior to screening, with a baseline HbA1c between 7.5 and 8.9, ⅔ of whom were on Metformin. Nevertheless, HbA1c levels and fructosamine levels decreased significantly and body weight decreased by 1.5 Kg. Postprandial responses were not determined. Side effects were not different from placebo. The drugs have a long safety record and NAC is used clinically as a detoxicant for free radical poisoning by acetaminophen.

For clinical use, the response to the test meal is tested twice. Either a placebo or BENAC, (SciMar NuPa Renew), are given at a proscribed time before consumption of a standardized, calibrated test meal (SciMarTest meal). Blood samples taken before and after the meal are compared to determine the meal-induced levels of insulin and glucose (MIG). A healthy MIG is seen as modest increases in both insulin and glucose because hepatalin action accounts for the majority of glucose uptake and is therefore insulin sparing. AMIS is the result of absence of hepatalin action resulting in increased levels of glucose and compensatory increases in insulin secretion. A MIG score can be calculated from different blood sampling times, preferably by the equation MIG=fed glucose×fed insulin−fasted glucose×fasted insulin. Postprandial sampling can be done between 60 and 120 minutes with a preferred standardized level based on the average postprandial levels taken at 60 and 90 minutes. If a poor MIG score is shown to improve after taking the drug, a diagnosis has been made and a therapy indicated. The chronic premeal therapy would use the same tablet that was used as the diagnostic tool to be used for chronic treatment.

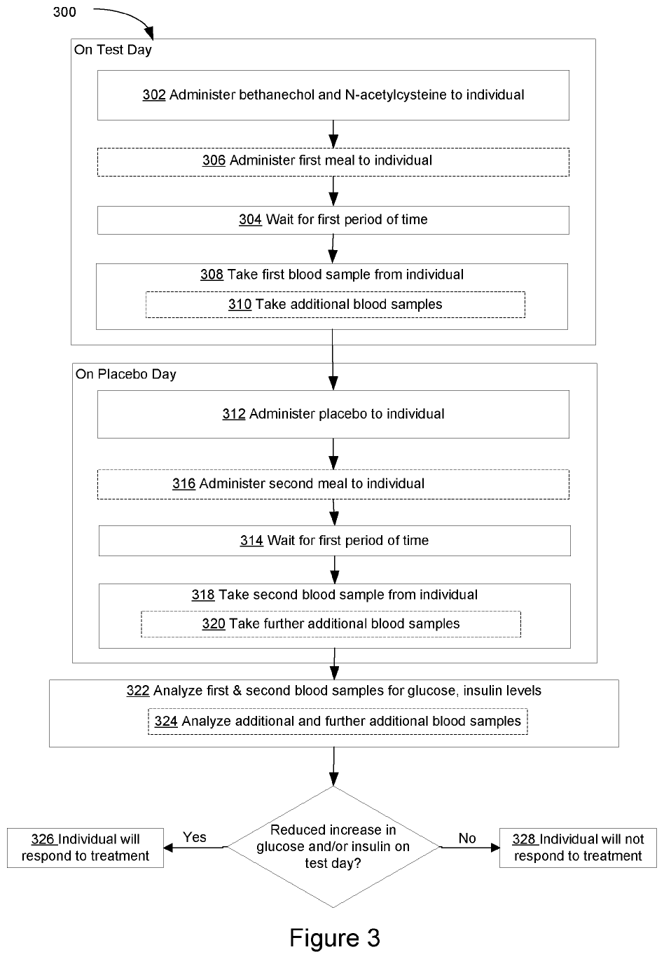

Thus, FIG. 3 illustrates an example method 300 for determining whether an individual with prediabetes, AMIS, type II diabetes, insulin resistance, or impaired glucose tolerance will respond to the bethanechol and N-acetylcysteine treatment, without medically treating the prediabetes, AMIS, type II diabetes, insulin resistance, or impaired glucose tolerance. Generally, method 300 comprises two rounds of testing.

On a first day (Round 1), at 302 OR 312, either (1) bethanechol and N-acetylcysteine or (2) a placebo is administered to the individual. In some applications, the amount of bethanechol and N-acetylcysteine administered may be 25 mg of bethanechol and 500 mg of N-acetylcysteine.

Administration of bethanechol and N-acetylcysteine or the placebo may be made by a variety of suitable routes including oral, topical (including transdermal, buccal or sublingal), nasal, inhalation, and parenteral (including intraperitoneal, subcutaneous, intravenous, intradermal or intramuscular injection) with oral or parenteral being generally preferred. The bethanechol and N-acetylcysteine may be administered together or separately, though together in a single formulation is preferred.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol, or cellulose preparations such as, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents may be added, such as the cross-linked polyvinylpyrrolidone, agar, or alginic acid or a salt thereof such as sodium alginate.

In a preferred embodiment, the bethanechol and N-acetylcysteine or the placebo may be administered orally in tablet form. It also will be appreciated that the preferred method of administration and dosage amount may vary with, for example, the condition and age of the recipient.

At 306, a first meal is administered to the individual. The first meal may be a consumable product that is constituted to be effective in activating Meal-Induced Insulin Sensitization (MIS) in humans/the individual. For example, the first meal may be a mixture consisting of proteins/amino acids, lipids and glucose/carbohydrates. In some applications, the first meal may comprise dextrose, lecithin, and soy protein.

Dextrose is a simple sugar that is made from corn and corresponds to glucose, or blood sugar. Since dextrose is a simple sugar, it can raise the individual's blood sugar level very quickly upon consumption. In one aspect, the first meal composition may comprise 80-85% by weight of dextrose. In a preferred embodiment, the amount of dextrose is 82.5% by weight.

Lecithin is a fat that is essential to the cells of the body. It is typically found in many foods, including soybeans and egg yolks. In one aspect, the first meal composition may comprise 2-8% by weight of lecithin. In a preferred embodiment, the amount of lecithin is 5% by weight.

Soy protein is a protein that is, unsurprisingly, isolated from soybean. It is made from soybean meal that has been dehulled and defatted. In one aspect, the first meal composition may comprise 10-15% by weight of soy protein. In a preferred embodiment, the amount of soy protein is 12.5% by weight.

Administration of the first meal may be made by a variety of suitable routes with oral being generally preferred. It also will be appreciated that the amount of the first meal administered to the individual may be proportional to the individual's body weight and/or may be varied based on the individual's condition and age.

At 308, a first blood sample is taken on the first day from the individual after a second period of time from when the first meal was administered to the individual. This second period of time allows the individual's body to react to the first meal that was administered at 306. In some applications, the second period of time may be between 30 and 180 minutes. In a preferred embodiment, the second period of time is 120 minutes. In other words, the first blood sample may be taken from the individual 120 minutes after the first meal was administered.

Optionally, at 310, additional blood samples may be taken from the individual on the first day at various times after the administration of the first meal at 306. For example, in some applications, four additional blood samples (i.e. in addition to the first blood sample) may be taken from the individual on the first day at four different times after the administration of the first meal. In a preferred embodiment, those various times may be 45 minutes, 60 minutes, 90 minutes, and 120 minutes after the administration of the first meal. Taking the additional blood samples at these additional various times allows for monitoring of how the individual's body processes the first meal over time.

The first and additional blood samples may be analyzed right away or may be analyzed after the second round of testing. The second round of testing is nearly identical to the first round of testing and takes place on another day (different from the first day). The other day may be a day that is between one to two weeks after the first day. In a preferred embodiment, the second round of testing takes place a week after the first round of testing.

Accordingly, on the other day (Round 2) at 312 or 302, the other one of bethanechol and N-acetylcysteine and the placebo (i.e. the composition that was not administered to the individual at 302) is administered to the individual. If bethanechol and N-acetylcysteine is administered, the amount may be 25 mg of bethanechol and 500 mg of N-acetylcysteine. The manner of administration of bethanechol and N-acetylcysteine or the placebo at 312 would be generally the same as the manner of administration at 302.

At 316, a second meal is then administered to the individual. The second meal may be a consumable product that is effective in activating Meal-Induced Insulin Sensitization (MIS) in humans/the individual. For example, the second meal may be a mixture consisting of proteins/amino acids, lipids and glucose/carbohydrates, such as dextrose, lecithin, and soy protein. It will be appreciated that the composition and the amount of the second meal administered to the individual at 316 would generally be the same as the first meal that was administered to the individual at 306.

Then at 314, method 300 may involve waiting for the first period of time. This first period of time allows the individual's body to process the bethanechol and N-acetylcysteine or the placebo that was administered at 312. In some applications, the first period of time may be between 30 and 180 minutes. Notably, the amount of time waited at 314 would generally be same as the amount of time waited at 304.

At 318, a second blood sample is taken on the other day from the individual after the second period of time from when the second meal was administered to the individual. This second period of time allows the individual's body to process at least some of the second meal that was administered at 316.

Optionally, at 320, further additional blood samples may be taken from the individual on the other day at the various times after the administration of the second meal at 316. For example, in some applications, four further additional blood samples (i.e. in addition to the second blood sample) may be taken from the individual on the other day at four different times after the administration of the second meal. Notably, the number of additional blood samples taken on the first day at 310 would be the same as the number of further additional blood samples taken on the other day at 320. As well, the additional blood samples (from the first day) and the further additional blood samples (from the other day) are generally taken at the same various times on the respective days after the corresponding first or second meal is administered. For example, as noted above, if the first and additional blood samples were taken on the first day (at 308 and 310) respectively 30 minutes, 45 minutes, 60 minutes, 90 minutes, and 120 minutes after the administration of the first meal, the second and further additional blood samples would be taken on the other day (at 318 and 320) respectively 30 minutes, 45 minutes, 60 minutes, 90 minutes, and 120 minutes after the administration of the second meal.

At 322, each of the first and second blood samples may be analyzed for one or more of a glucose level, and an insulin level. If additional blood samples and further additional blood samples were taken, they may also be analyzed at 324 for one or more of a glucose level and an insulin level.

The identified glucose and insulin levels of each of the blood samples are then compared. In particular a test increase in glucose and/or insulin between the first blood sample on the test day and the second blood sample on the test day is measured. A placebo increase in glucose and/or insulin between the first blood sample on the placebo day and the second blood sample on the placebo day is measured. A reduced increase in glucose and/or insulin on the test day as compared to the placebo day indicates that the individual will respond to the bethanechol and N-acetylcysteine treatment (at 326). If there is no difference in the respective glucose levels and the insulin levels, between the placebo day and the test day, this indicates that the individual will not be responsive to the bethanechol and N-acetylcysteine treatment (at 328).

The diagnostic thus involves the response to 2 test meals with one proceeded by a placebo tablet and one by a tablet containing bethanechol and N-acetylcysteine, applied randomly. One blood sample is determined before the meal and at times up to 120 minutes after the meal. If the drug resulted in a reduced postprandial increase in glucose and insulin over a period of time, such as 120 minutes, the diagnostic indicates that the drug could be beneficially used chronically.

In some applications, a difference of at least about 20% in the increase in glucose levels between the test day and the placebo day indicates that the individual will be responsive to the bethanechol and N-acetylcysteine treatment.

In some applications, a difference of at least about 20% in the increase in insulin levels between the test day and the placebo day indicates that the individual will be responsive to the bethanechol and N-acetylcysteine treatment.

If additional blood samples and further additional blood samples were taken at 310 and 320 and analyzed at 324, the corresponding glucose and/or insulin levels of the additional blood samples and further additional blood samples may also be compared.

The present disclosure also relates to a diagnostic kit for performing method 300. The diagnostic kit may be used with blood sample drawing equipment and blood analysis equipment. To that end, the diagnostic kit may include a first composition that comprises bethanechol and N-acetylcysteine and a second composition that comprises an inactive substance, where the second composition is visually identical to the first composition (i.e. a placebo). The state or form of the first and second compositions may vary depending on their method of administration to the individual. For example, the first composition may comprise 25 mg of bethanechol and 500 mg of N-acetylcysteine in tablet form for oral administration. The placebo would then also be in an identical tablet form. In a preferred embodiment, the bethanechol and N-acetylcysteine would be formulated into a single tablet, and the placebo would be formulated into an identical single tablet. It will be appreciated that the preferred method of administration and dosage amount may vary with, for example, the condition and age of the recipient.

The diagnostic kit may also include a first meal and a second meal, where the two meals are generally the same. The first and second meals may be a consumable product that is constituted to be effective in activating Meal-Induced Insulin Sensitization (MIS) in an individual. For example, in a preferred embodiment, the first and second meals may each comprise dextrose, lecithin, and soy protein, where dextrose is 82.5% of the meal by weight, lecithin is 5% of the meal by weight, and soy protein is 12.5% of the meal by weight. It also will be appreciated that the amount and composition of the first and second meals may be proportional to the individual's body weight and/or may be varied based on the individual's condition and age.

The diagnostic kit may further include instructions for the individual to carry out substantially the methods taught herein. In some applications, the diagnostic kit may further include equipment for drawing the blood samples, such as gloves, alcohol or iodine to cleanse the area, a tourniquet, tubes, a tube holder, needles, tape, and gauze.

Embodiments of the present invention are further described with reference to the following examples, which are intended to be illustrative and not limiting in nature.

### Example Study

40 subjects with a BMI greater than 28, HbA1c greater than 7 are selected. Those 40 subjects consist of 20 males and 20 females (where 10 are pre-ovulation and 10 are post-ovulation. The subjects are selected to have a high likelihood of a degree of AMIS. A range in level of dysfunctions will allow correlation analysis to be done. N=40 provides sufficient data to detect if causes of AMIS are related. Rapid Insulin Sensitivity Test (RIST) score are reduced in human subjects with BMI 27.2 compared with 22.7.

During Visit 1: each subject is screened by taking a blood sample. Each blood sample is analyzed for glucose, insulin, triglycerides, HbA1c, BMI, Body composition, with standard health assays.

During Visit 2: a baseline blood sample is taken prior to any administration. Then either a placebo or the bethanechol and N-acetylcysteine is given to each subject, where the administration of the placebo or bethanechol and N-acetylcysteine is randomized, and double blinded for each subject. After 30 minutes, each subject consumes a meal. Following consumption of the meal, blood samples are taken 30, 45, 60, 90, and 120 minutes after the meal. Each blood sample is then analyzed for glucose, insulin, triglycerides, and lactate levels.

During Visit 3: the same protocol as Visit 2 is performed for each subject but with the drug or placebo that was not administered during Visit 2. Thus, another baseline blood sample is taken. Then the placebo or the bethanechol and N-acetylcysteine that was not given to the subject during Visit 2 is given to each corresponding subject, where the administration continues to be double blinded. After 30 minutes, each subject consumes another of the same meal. Following consumption of the meal, another round of blood samples are taken 30, 45, 60, 90, and 120 minutes after the meal. Each blood sample in the second round is then analyzed for glucose, insulin, triglycerides, and lactate levels.

Throughout the description, specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such moadifications, permutations, additions and sub-combinations as are consistent with the broadest interpretation of the specification as a whole.

### REFERENCES

Xie, H and Lautt, W W, 1996, Endocrinology and Metabolism, Vol 270(5), E858-E863
Brownlee M (2001). Biochemistry and molecular cell biology of diabetic complications. Nature 414, 813-819.
Lautt, W. W., Ming, Z., Legare, D. J., 2010. Attenuation of age- and sucrose-induced insulin resistance and syndrome X by a synergistic antioxidant cocktail: the AMIS syndrome and HISS hypothesis. Can. J. Physiol. Pharmacol. 88(3), 313-323. https://doi.orq/10.1139/Y09-130.
Patarrao et al., 2008, Can. J. Physiol. Pharmacol. 86: 880-888. Lautt et al., 2011, Can. J. Physiol. Pharmacol. 89: 135-142.
Lautt, W. W., Wang, H. H., 2014. EP2803362A1. Hepatic insulin sensitizing substance and test meal for insulin sensitization. European Patent Application #14168369.8, published 19 Nov. 2014, bulletin 2014/47.

1. A method for determining whether an individual with prediabetes, AMIS, type 2 diabetes, gestational diabetes, insulin resistance, or impaired glucose tolerance will respond to a bethanechol and N-acetylcysteine treatment, the method comprising:
on a test day:
taking a first blood sample on the test day from the individual;
administering bethanechol and N-acetylcysteine to the individual;
administering a meal to the individual;
waiting a period of time; and
taking a second blood sample on the test day from the individual;
on a placebo day:
taking a first blood sample on the placebo day from the individual; administering a placebo to the individual;
administering the meal to the individual;
waiting the period of time; and
taking a second blood sample on the placebo day from the individual;
analyzing each of the blood samples for one or more of a glucose level and an insulin level;
quantifying a test increase in glucose and/or insulin between the first blood sample on the test day and the second blood sample on the test day;
quantifying a placebo increase in glucose and/or insulin between the first blood sample on the placebo day and the second blood sample on the placebo day;
wherein a reduced increase in glucose and/or insulin on the test day as compared to the placebo day indicates that the individual will respond to the bethanechol and N-acetylcysteine treatment.

2. The method of claim 1, wherein the reduced increase in glucose is a reduction of at least about 20%.

3. The method of claim 1, wherein the reduced increase in insulin is a reduction of at least about 20%.

4. The method of claim 1, wherein about 25 mg of the bethanechol and about 500 mg of the N-acetylcysteine is administered.

5. The method of claim 1, wherein the period of time is between about 30 and about 180 minutes.

6. The method of claim 5, wherein the period of time is about 120 minutes.

7. The method of claim 1, wherein the meal comprises dextrose, lecithin, and soy protein.

8. The method of claim 1, further comprising taking additional test blood samples on the test day from the individual at various times after the administration of the meal, and further comprising taking additional placebo blood samples on the placebo day from the individual at the various times after the administration of the meal.

9. The method of claim 8, wherein the number of additional test blood samples is the same as the number of additional placebo blood samples.

10. The method of claim 9, wherein the number of additional test blood samples and the number of additional placebo blood samples is four.

11. The method of claim 8, wherein the various times are 45 minutes, 60 minutes, 90 minutes, and 120 minutes after the administration of the meal.

12. The method of claim 1, wherein the placebo day is between one and two weeks before or after the test day.

13. (canceled)

14. A diagnostic kit for use with blood sample drawing equipment and blood analysis equipment for diagnosing whether an individual with prediabetes, AMIS, type 2 diabetes, insulin resistance, or impaired glucose tolerance will respond to a bethanechol and N-acetylcysteine treatment according to the method of claim 1, the kit comprising:
a test composition comprising the bethanechol and the N-acetylcysteine;
the placebo composition comprising an inactive substance; and
at least two of the meal.

15. The diagnostic kit of claim 14, comprising about 25 mg of the bethanechol and about 500 mg of the N-acetylcysteine.

16. The diagnostic kit of claim 14, wherein the test composition and the placebo composition are in tablet form.

17-19. (canceled)

20. The diagnostic kit of claim 14, wherein the meal further comprises dextrose, lecithin, and soy protein.

21. The diagnostic kit of claim 20, wherein the meal comprises about 82.5% of dextrose by weight, about 5% of lecithin by weight, and about 12.5% of soy protein by weight.

22. The diagnostic kit of claim 14, further comprising instructions to carry out the method.

23. The diagnostic kit of claim 14, further comprising blood sample drawing equipment.

24. A method of treating one or more of prediabetes, AMIS, type 2 diabetes, gestational diabetes, insulin resistance, or impaired glucose tolerance in an individual in need thereof, said-use comprising determining whether the individual with prediabetes, AMIS, type 2 diabetes, gestational diabetes, insulin resistance, or impaired glucose tolerance will respond to a bethanechol and N-acetylcysteine treatment by applying the method ofany of claim 1 to the individual, wherein if application of the method of claim 1 to the individual indicates that the individual will respond to the bethanechol and the N-acetylcysteine treatment, the bethanechol and the N-acetylcysteine are administered to the individual.
